(19) United States
(12) Patent Application Publication
(10) Pub. No.: US 2026/0233029 A1
(43) Pub. Date: Aug. 13, 2026

(54) DOSE MANAGEMENT APPARATUS, DOSE MANAGEMENT METHOD, AND STORAGE MEDIUM

(71) Applicant: Konica Minolta, Inc., Tokyo (JP)

(72) Inventors: Yuki TOMIYAMA, Tokyo (JP); Kazuyuki MAEDA, Tokyo (JP)

(21) Appl. No.: 19/530,504

(22) Filed: Feb. 5, 2026

(30) Foreign Application Priority Data
Feb. 12, 2025 (JP) 2025-020783

(51) Int. Cl. A61N 5/10; G16H 20/40

(52) U.S. Cl. CPC A61N 5/1031; G16H 20/40

(57) ABSTRACT

A dose management apparatus manages radiation dose information on an examination of a subject and includes a hardware processor that records whether an explanation about gonadal protection has been made for execution of an examination. In one embodiment, the hardware processor records whether the explanation about the gonadal protection has been made before the execution of the examination.

### CROSS-REFERENCE TO RELATED APPLICATION

The entire disclosure of Japanese Patent Application No. 2025-020783, filed on Feb. 12, 2025, including description, claims, drawings and abstract is incorporated herein by reference.

### BACKGROUND OF THE INVENTION

### Technical Field

The present disclosure relates to a dose management apparatus, a dose management method, and a storage medium.

### Description of Related Art

In recent years, with improvement in performance of a radiographic imaging apparatus, it has been discussed to make it unnecessary to use a gonadal protector for children. However, due to the practice that gonadal protectors have been used for children for the past seventy years, it is expected that patients and their guardians will feel uneasy when receiving radiographic imaging without using gonadal protectors. In order to eliminate such a sense of anxiety, it is assumed that it is necessary to explain to the patient or his/her guardian that there is no problem without a gonadal protective garment. In addition, as a technique for managing information on an exposure dose, a dose management apparatus that manages dose information on an emission dose of an emission event in which a radiographic image is generated in response to radiation emission to a subject has been proposed (see Japanese Unexamined Patent Publication No. 2021-186036).

### SUMMARY OF THE INVENTION

The conventional technique can present dose information to a patient or his/her guardian, but cannot manage as history information whether or not it has been explained to the patient or the like that a gonadal protector is unnecessary.

Therefore, in order to solve the above-described problem, an object of the present disclosure is to provide a dose management apparatus, a dose management method, and a storage medium storing a program that are capable of managing whether or not an explanation about gonadal protection has been given to a patient or the like in a case of performing a radiographic imaging examination.

To achieve at least one of the abovementioned objects, according to an aspect of the present disclosure, a dose management apparatus reflecting one aspect of the present disclosure manages radiation dose information on an examination of a subject and includes a hardware processor that records whether an explanation about gonadal protection has been made for execution of an examination.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a diagram showing an example of a schematic configuration of a medical information management system according to the present embodiment;

FIG. 2 is a block diagram of a dose management apparatus according to the present embodiment;

FIG. 3 is a block diagram of a client terminal according to the present embodiment;

FIG. 4 is a view for explaining the flow of a process of registering image data of a radiographic image and an RDSR generated by an examination apparatus according to the present embodiment;

FIG. 5 is a flowchart illustrating an example of the operation of the medical information management system in the case of recording, in the dose management apparatus, information on an explanation about gonadal protection input on an explanation information input screen of the client terminal according to the present embodiment;

FIG. 6 is a view illustrating an example of the explanation information input screen displayed on a display part of the client terminal according to the present embodiment;

FIG. 7 is a flowchart illustrating an example of the operation of the medical information management system in the case of displaying, on the display part of the client terminal, information on the explanation about gonadal protection recorded in the dose management apparatus according to the present embodiment;

FIG. 8 is a view illustrating an example of a dose information management screen displayed on the display part of the client terminal according to the present embodiment; and

FIG. 9 is a view illustrating an example of a gonadal protection explanation information screen displayed on the display part of the client terminal according to the present embodiment.

### DETAILED DESCRIPTION

Hereinafter, one or more embodiments of the present disclosure will be described with reference to the drawings. However, the scope of the invention is not limited to the disclosed embodiments.

Hereinafter, with reference to the accompanying drawings, a dose management apparatus, a dose management method, and a program according to a preferred embodiment(s) of the present disclosure will be described.

### Example of Configuration of Medical Information Management System 1

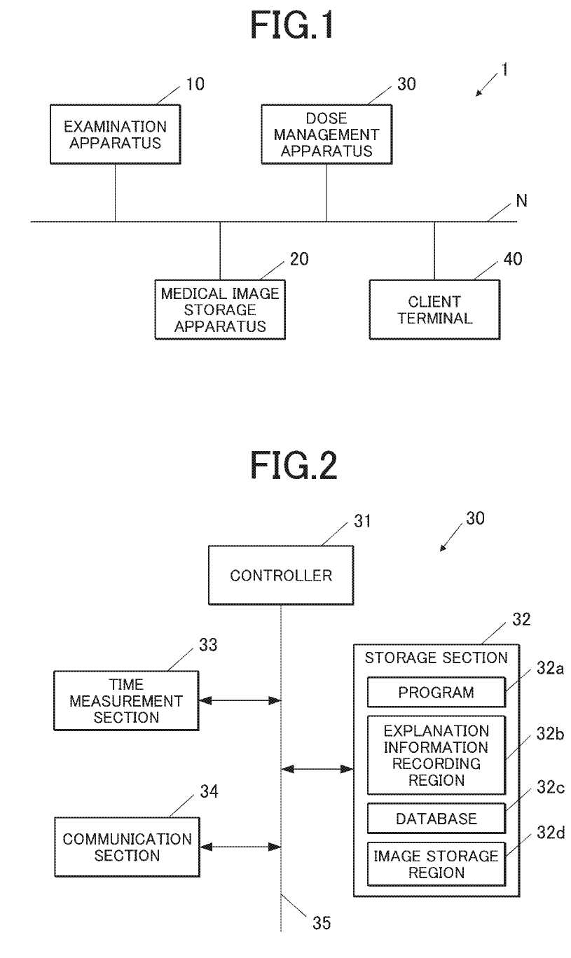

FIG. 1 is a diagram showing an example of a schematic configuration of a medical information management system 1 according to the present embodiment. The medical information management system 1 includes an examination apparatus 10, a medical image storage apparatus 20, a dose management apparatus 30, and a client terminal 40. The examination apparatus 10, the medical image storage apparatus 20, the dose management apparatus 30, and the client terminal 40 are communicably connected to each other via a network N. Examples of the network N include a LAN, a WAN, and the Internet. LAN is an abbreviation for Local Area Network. WAN is an abbreviation for Wide Area Network. Each apparatus such as the examination apparatus 10 conforms to the DICOM standard, and performs communication in accordance with the DICOM standard. DICOM is an abbreviation for Digital Image and Communications in Medicine. Note that the numbers of examination apparatuses 10, medical image storage apparatuses 20, dose management apparatuses 30 and client terminals 40, which constitute the medical information management system 1, are not limited to the numbers thereof illustrated in FIG. 1.

The examination apparatus 10 is, for example, an X-ray imaging apparatus such as a DR or a CR. The examination apparatus 10 operates in response to operations made by a radiological technologist or the like on a console (not illustrated). The examination apparatus 10 irradiates a subject (patient) with radiation (i.e., emits radiation to a subject), and generates image data of a radiographic image as a medical image on the basis of a detection result of the radiation. The examination apparatus 10 writes supplementary information into a header of the image data in accordance with the DICOM standard, to generate a DICOM image file with the supplementary information is added to the radiographic image.

The supplementary information includes, for example, patient information, examination information, series information, and image information. The patient information is information on a patient. The patient information includes, for example, a patient ID, a patient name, a date of birth, a gender, a height, a weight, an age, a BMI, and the like. The examination information is information on an examination. The examination information includes, for example, an examination ID, an examination date, an examination time, an examination description, and an examination instance UID. The series information is information on a series. The series information includes, for example, a series instance UID, a series number, a series date, a series time, a modality (DR, CR, etc.), and a series description. The image information is information on an image. The image information includes, for example, an SOP instance UID, an image date, an image time, and an image number.

The examination apparatus 10 generates, for each radiation emission event, an RDSR including dose information on the examination performed in the examination apparatus 10. RDSR is an abbreviation for Radiation Dose Structured Report. The examination apparatus 10 transmits the generated image file of the radiographic image and the generated RDSR to the medical image storage apparatus 20 and the dose management apparatus 30. Note that the RDSR is information compliant with the DICOM standard, and is one of data formats of data including radiation dose information. The dose information is information on a radiation emission dose emitted to the subject in a radiological examination. The dose information may include information on an amount correlated with the emission dose, such as the voltage and current applied for radiation emission and the radiation emission time. The emission event is a group of radiation emission operations to the subject, usually a series of emission operations under the same emission condition(s), and in each emission event, one or more radiographic images are generated in response to the radiation emission.

The medical image storage apparatus 20 stores and manages, for each patient and each examination, the image file of the radiographic image generated by the examination apparatus 10 and the dose information included in the RDSR. Examples of the medical image storage apparatus 20 include a PACS. PACS is an abbreviation for Picture Archiving and Communication System.

The dose management apparatus 30 is a computer apparatus for storing and managing a radiographic image of a subject, dose information, information on an explanation about gonadal protection, and the like at the time of an examination. Examples of the case where the recording of the information on the explanation about the gonadal protection is required includes a case where radiographic imaging is performed on a child (subject) of 12 years old or younger for whom the use of a gonadal protector is conventionally required. Note that details of the dose management apparatus 30 will be described later. In addition, hereinafter, the information on the explanation about the gonadal protection may be referred to as gonadal protection explanation information.

The client terminal 40 is a computer apparatus that is used by a user such as a doctor or a radiological technologist. Examples of the client terminal 40 include a personal computer and a tablet. The client terminal 40 acquires, from the dose management apparatus 30, a radiographic image, dose information, gonadal protection explanation information, and the like at the time of an examination on a patient, in response to a display request from the user, and displays these pieces of information on the screen. For example, the user can view the gonadal protection explanation information and the like by using a web browser, a viewer function, or the like of the client terminal 40.

### Example of Configuration of Dose Management Apparatus 30

FIG. 2 is a block diagram of the dose management apparatus 30 according to the present embodiment. The dose management apparatus 30 includes a controller 31 (hardware processor), a storage section 32, a time measurement section 33, and a communication section 34. The controller 31, the storage section 32, the time measurement section 33, and the communication section 34 are connected to each other by wiring 35 such as a bus. Note that the configuration of the dose management apparatus 30 is not limited to that shown in FIG. 2, but may include, for example, a display part, an operation part, and the like, similarly to the client terminal 40 to be described later.

The controller 31 includes, for example, a processor, such as a CPU, having an arithmetic function, a control function, and the like, and a memory such as a RAM. CPU is an abbreviation for Central Processing Unit. RAM is an abbreviation for Random Access Memory. The controller 31 executes programs 32a (described later) stored in the memory such as a RAM, the storage section 32, or the like, thereby controlling the entire apparatus and also realizing various functions, for example, to manage various kinds of information such as radiographic images and radiation dose information.

In the present embodiment, the controller 31 functions as a recording section which records whether or not the explanation about gonadal protection has been given to the patient, his/her guardian or the like at the time of examination by radiographic imaging. Specifically, the dose management apparatus 30 performs control to record the following gonadal protection explanation information.
(1) Information indicating whether the explanation about gonadal protection has been made for the execution of the examination
(2) Information indicating whether the explanation about gonadal protection has been made before and/or after the examination
(3) Information indicating whether the explanation about gonadal protection has been given to a person other than the subject and a relationship between the person who has received the explanation and the subject
(4) Information indicating the contents of the explanation about gonadal protection
(5) Information indicating comments on execution (making/giving) of the explanation about gonadal protection
Note that as the gonadal protection explanation information recorded in the dose management apparatus 30, the above-described information (1) may be essential, and the above-described information (2) to (5) may be optional. Further, the gonadal protection explanation information may include information other than the information exemplified in the above (1) to (5).

Furthermore, in the present embodiment, the controller 31 functions as an output section that outputs the gonadal protection explanation information recorded by the recording section. Specifically, the output section outputs the gonadal protection explanation information and the dose information on the exposure dose in the examination to a display device provided in the client terminal 40 or the like. The information on the exposure dose includes a reference value of the exposure dose for the age and weight of the subject. The reference value of the exposure dose is determined based on the incident surface dose or the area dose.

The storage section 32 includes, for example, at least one of storage modules such as an HDD, an SSD, a ROM, and a RAM. HDD is an abbreviation for Hard Disk Drive. SSD is an abbreviation for Solid State Drive. ROM is an abbreviation for Read Only Memory. The storage section 32 stores, for example, programs 32a capable of managing radiographic images, date, dose information, and the like obtained by each examination. Furthermore, the storage section 32 is provided with an explanation information recording region 32b which functions as a recording section, a database 32c, and an image storage region 32d. In the explanation information recording region 32b, the gonadal protection explanation information in the examination of each subject shown in (1) to (5) described above is recorded based on the control of the controller 31. The explanation information recording region 32b may be linked to the database 32c and the image storage region 32d, and may be recorded in a searchable manner. The explanation information recording region 32b may be incorporated in the database 32c or the like.

The database 32c stores, in a searchable manner, supplementary information on files of radiographic images stored in the image storage region 32d, emission event information including dose information on each examination, and the like. For example, the database 32c includes a patient information table, an examination information table, a series information table, an image information table, and an emission event information table. The image storage region 32d stores, for example, image data of a radiographic image(s) of each subject captured at the time of examination. Each image data is stored in a folder that is hierarchized in the order of date, patient ID, examination instance UID, and series instance UID. As the contents stored in the database 32c, for example, the contents of a database known by Japanese Unexamined Patent Publication No. 2021-186036 or the like can be adopted. In the image storage region 32d, by dividing the folders by date, by patient, by examination, and by series, high-speed access can be achieved when a display instruction for each screen is acquired.

The time measurement section 33 includes a timing circuit. The timing circuit is referred to as RTC, and RTC is an abbreviation for Real Time Clock. The time measurement section 33 performs time measurement of the current date and time with the timing circuit and outputs the current date and time to the controller 31.

The communication section 34 includes, for example, a communication module such as an NIC or a transmitter/receiver. NIC is an abbreviation for Network Interface Card. The communication section 34 transmits and receives various kinds of information and data to and from the examination apparatus 10, the medical image storage apparatus 20, and the client terminal 40 via the network N.

### Example of Configuration of Client Terminal 40

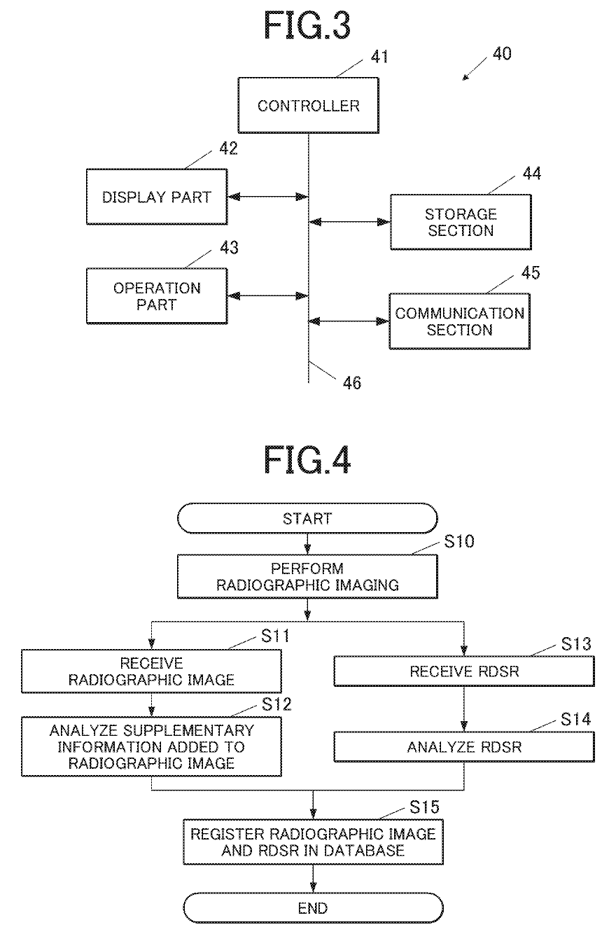

FIG. 3 is a block diagram of the client terminal 40 according to the present embodiment. The client terminal 40 includes a controller 41, a display part 42, an operation part 43, a storage section 44, and a communication section 45. The controller 41, the display part 42, the operation part 43, the storage section 44, and the communication section 45 are connected to each other by wiring 46 such as a bus.

The display part 42 is, for example, a display device such as a liquid crystal display or an organic EL display. EL is an abbreviation for Electro Luminescence. The display part 42 displays, on its screen, a GUI or the like for receiving various input operations from the user. GUI is an abbreviation for Graphical User Interface. The display part 42 displays, for example, a radiographic image, dose information, information on the explanation about gonadal protection, and the like at the time of examination of the subject.

The operation part 43 includes, for example, at least one of a mouse, a keyboard, a switch, a button, a sensor, and the like. The operation part 43 may be, for example, a touch screen integrally combined with a display. The operation part 43 receives an instruction corresponding to a user's input operation, and outputs operation information based on the received instruction to the controller 41. The operation part 43 receives, for example, an instruction for recording, in the dose management apparatus 30, information on the explanation about gonadal protection at the time of examination of the subject, for example.

The storage section 44 includes, for example, at least one of storage modules such as an HDD, an SSD, a ROM, and a RAM. HDD is an abbreviation for Hard Disk Drive. SSD is an abbreviation for Solid State Drive. ROM is an abbreviation for Read Only Memory. The storage section 44 stores, for example, a system program, application programs, and various types of data.

The communication section 45 includes, for example, a communication module such as an NIC or a transmitter/receiver. NIC is an abbreviation for Network Interface Card. The communication section 45 transmits and receives various kinds of information and data to and from the examination apparatus 10, the medical image storage apparatus 20, and the dose management apparatus 30 via the network N.

### Registration Process of Image Data of Radiographic Image and RDSR

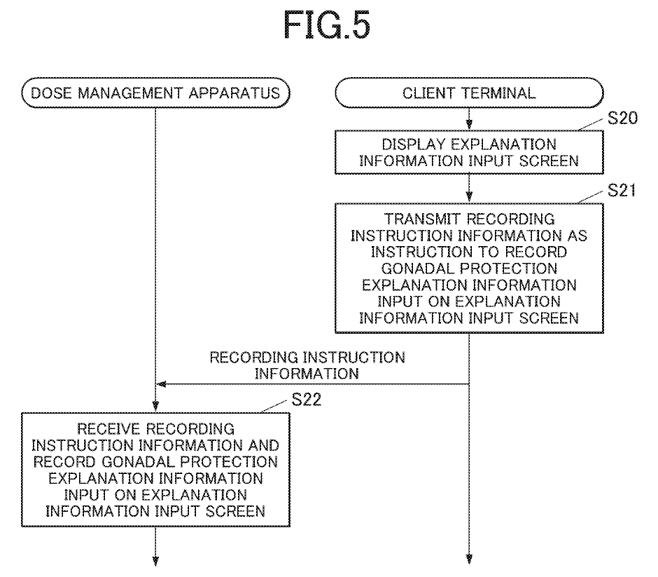

FIG. 4 is a view for explaining the flow in a case where image data of a radiographic image and an RDSR generated by the examination apparatus 10 are recorded according to the present embodiment are registered. The examination apparatus 10 acquires image data of a radiographic image by performing radiographic imaging on the subject based on an occurred emission event (Step S10). The examination apparatus 10 transmits the acquired image data of the radiographic image and the RDSR related to the emission event to the dose management apparatus 30. The image data of the radiographic image and the RDSR related to the emission event may be transmitted to the medical image storage apparatus 20.

The communication section 34 of the dose management apparatus 30 receives the image data of the radiographic image from the examination apparatus 10. The controller 31 acquires the image data of the radiographic image received by the communication section 34 (Step S11). The controller 31 analyzes the supplementary information added to the radiographic image for each acquired radiographic image (Step S12). Specifically, the controller 31 acquires, for each item, patient information, examination information, series information, image information, and the like from the supplementary information on the radiographic image.

The communication section 34 of the dose management apparatus 30 receives the RDSR from the examination apparatus 10 in parallel with the reception process of the image data of the radiographic image. The controller 31 acquires the RDSR received by the communication section 34 (Step S13). The controller 31 analyzes the acquired RDSR (Step S14). Specifically, the controller 31 acquires, for each item, patient information such as a patient ID, examination information, dose information, and the like from the RDSR.

The controller 31 associates and records the dose information included in the acquired RDSR with the radiographic image generated in the emission event corresponding to the dose information in the storage section 32 (Step S15). In the above-described data registration process, the dose management apparatus 30 receives the radiographic image and the RDSR from the examination apparatus 10, but the dose management apparatus 30 may receive the radiographic image and the RDSR from the medical image storage apparatus 20. Further, in the above, the dose management apparatus 30 receives and analyzes the radiographic image and the RDSR in parallel from the examination apparatus 10 or the like, but the radiographic image and the RDSR may be received and analyzed in series.

### Example of Operation of Medical Information Management System 1 in Case of Registering Gonadal Protection Explanation Information

FIG. 5 is a flowchart illustrating an example of the operation of the medical information management system 1 in a case where gonadal protection explanation information input on the explanation information input screen of the client terminal 40 is recorded in the dose management apparatus 30 according to the present embodiment.

A user such as a radiological technologist or a doctor explains to the subject or his/her guardian that the use of a gonad protector is unnecessary, at least one of the times that are before an examination in which radiographic imaging of the subject is performed and after the examination in which radiographic imaging of the subject is performed. The explanation that the use of a gonadal protector is unnecessary can be provided, for example, by displaying an explanation text, a photograph, a figure, a graph, or the like indicating that the use of a gonadal protector is unnecessary on the display part 42 of the client terminal 40. The explanation that the use of a gonadal protector is unnecessary may be displayed on a display part of a console (not shown) of the examination apparatus 10. Further, the explanation that the use of a gonadal protector is unnecessary may be made orally by the radiological technologist or the like, or may be made using a pamphlet or the like.

When the user finishes explaining to the patient or the like that a gonadal protection garment is unnecessary, the user makes an operation to display the explanation information input screen on the display part 42 in order to record, as history information, that the explanation that a gonadal protection garment is unnecessary has been made. For example, if the user explains before a radiographic imaging examination that a gonadal protection garment is unnecessary, the user may input the gonadal protection explanation information before the examination or may input the gonadal protection explanation information after the examination. If the user explains that a gonadal protection garment is unnecessary both before and after the radiographic imaging examination, the user may input pieces of the gonadal protection explanation information collectively after the examination, or may input pieces of the gonadal protection explanation information before and after the examination, respectively.

Based on a display instruction from the user, the controller 41 of the client terminal 40 displays the explanation information input screen on the display part 42 (Step S20). For example, the controller 41 controls the communication section 45 or the like to connect to the dose management apparatus 30 via the web browser, thereby displaying the explanation information input screen on the display part 42. The information on the explanation information input screen is stored in the dose management apparatus 30 and corresponds to an example of an input section. The explanation information input screen is provided with, for example, an input section for inputting various kinds of information indicating that the explanation that the use of a gonadal protector is unnecessary has been made.

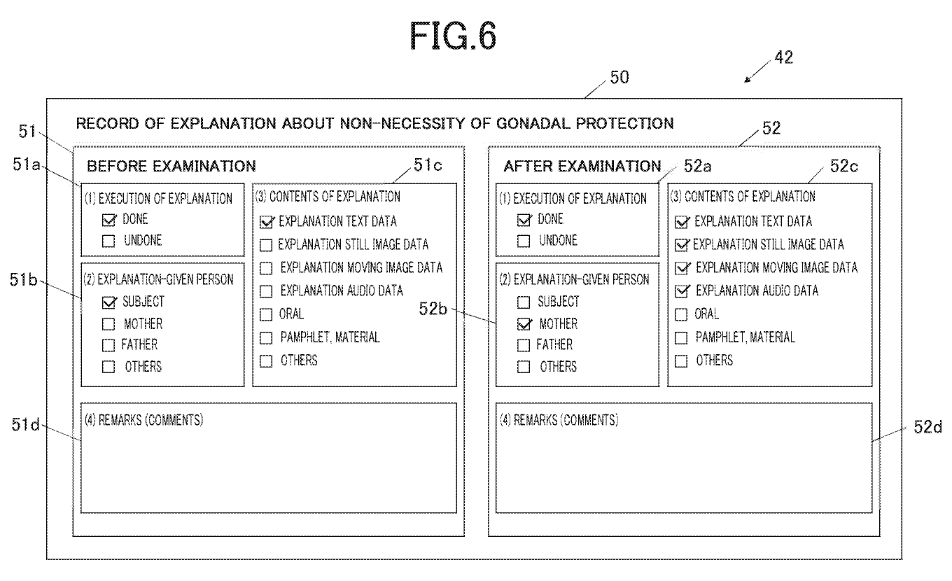

FIG. 6 is a view illustrating an example of an explanation information input screen 50 (inputter) displayed on the display part 42 of the client terminal 40 according to the present embodiment. The explanation information input screen 50 includes a pre-examination input section 51 for making an input if the explanation that the use of a gonadal protector is unnecessary is made before an examination, and a post-examination input section 52 for making an input if the explanation that the use of a gonadal protector is unnecessary is made after the examination. Since the pre-examination input section 51 and the post-examination input section 52 have substantially the same contents, the configuration of the pre-examination input section 51 will be representatively described below.

To be specific, the pre-examination input section 51 is provided with an explanation execution input section 51a for inputting whether the explanation about gonadal protection has been done. In the explanation execution input section 51a, “done” or “not done” can be selected. The pre-examination input section 51 is provided with a target input section 51b for inputting whether or not the explanation about gonadal protection has been given to a person other than the subject and the relationship between the person who has received the explanation and the subject. In the target input section 51b, “subject”, “mother”, “father”, and “others” can be selected. The pre-examination input section 51 is provided with a content input section 51c for inputting information used at the time of the explanation about gonadal protection. In the content input section 51c, “explanation text data”, “oral”, “pamphlet”, “others” and the like can be selected. The pre-examination input section 51 is provided with a comment input section 51d for inputting comments such as a situation at the time of giving the explanation about gonadal protection to the subject or the like. Similarly to the pre-examination input section 51, the post-examination input section 52 is provided with an explanation execution input section 52a, a target input section 52b, a content input section 52c, and a comment input section 52d. In FIG. 6, each input section of each of the pre-examination input section 51 and the post-examination input section 52 is configured by checkboxes, but not limited thereto. Each input section thereof may be configured by, for example, a pull-down list.

In a case where the user such as the radiological technologist has explained to the subject that a gonadal protector is unnecessary by using text data on the screen before the examination, as shown in FIG. 6, the user selects “done” in the explanation execution input section 51a of the pre-examination input section 51. Further, the radiological technologist or the like selects “subject” in the target input section 51b of the pre-examination input section 51, and selects “explanation text data” in the content input section 51c. Similarly, in a case where the user has explained to the subject that a gonadal protector is unnecessary after the examination, as shown in FIG. 6, the user selects an item concerned in each input section of the post-examination input section 52.

When finishing the input of the gonadal protection explanation information on the explanation information input screen 50, the user gives an instruction to record the input gonadal protection explanation information in the dose management apparatus 30. The controller 41 of the client terminal 40 transmits recording instruction information corresponding to a user’s recording operation received on the explanation information input screen 50 or the like to the dose management apparatus 30 via the network N (Step S21).

When the controller 31 of the dose management apparatus 30 receives the recording instruction information from the client terminal 40 through the communication section 34, the controller 31 records, in the storage section 32, the gonadal protection explanation information input in each input section of the explanation information input screen 50 (Step S22). For example, the controller 31 records, in the storage section 32, the gonadal protection explanation information such as “done” and “subject” input in each item under “before examination” and “after examination” on the explanation information input screen 50 shown in FIG. 6 in association with the corresponding radiographic image, RDSR, and the like of the subject. Note that Step S22 corresponds to an example of a recording step.

### Example of Operation of Medical Information Management System 1 in Case of Displaying Gonadal Protection Explanation Information

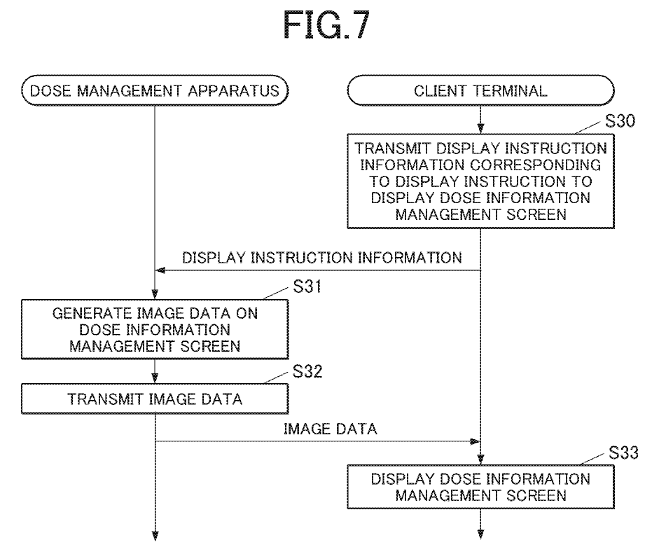

FIG. 7 is a flowchart illustrating an example of the operation of the medical information management system 1 in a case where the gonadal protection explanation information or the like recorded in the dose management apparatus 30 is displayed on the display part 42 of the client terminal 40 according to the present embodiment.

In a case where a user such as a radiological technologist or a doctor checks dose information and gonadal protection explanation information at the time of examination of a predetermined subject, the user operates the operation part 43 to input a display instruction for displaying a dose information management screen. For example, the display instruction may be input on a menu screen or the like of the dose management apparatus 30. The dose information management screen is a screen for displaying in detail dose information and the like in an emission event of the subject. When a user’s display instruction/operation is received on the screen of the display part 42, the controller 41 of the client terminal 40 transmits display instruction information corresponding to the display operation to the dose management apparatus 30 via the network N (Step S30).

When the controller 31 of the dose management apparatus 30 receives the display instruction information or the like from the client terminal 40 through the communication section 34, the controller 31 generates image data on a dose information management screen for the subject (Step S31). For example, the controller 31 reads the dose information, the radiographic image, and the gonadal protection explanation information associated with the target subject from the storage section 32, and generates image data on the dose information management screen. In this case, the controller 31 may use the patient ID of the subject or the like to search the explanation information recording region 32b, the database 32c, the image storage region 32d, and/or the like.

The controller 31 of the dose management apparatus 30 controls the communication section 34 to transmit the image data on the dose information management screen for the subject to the client terminal 40 via the network N (Step S32).

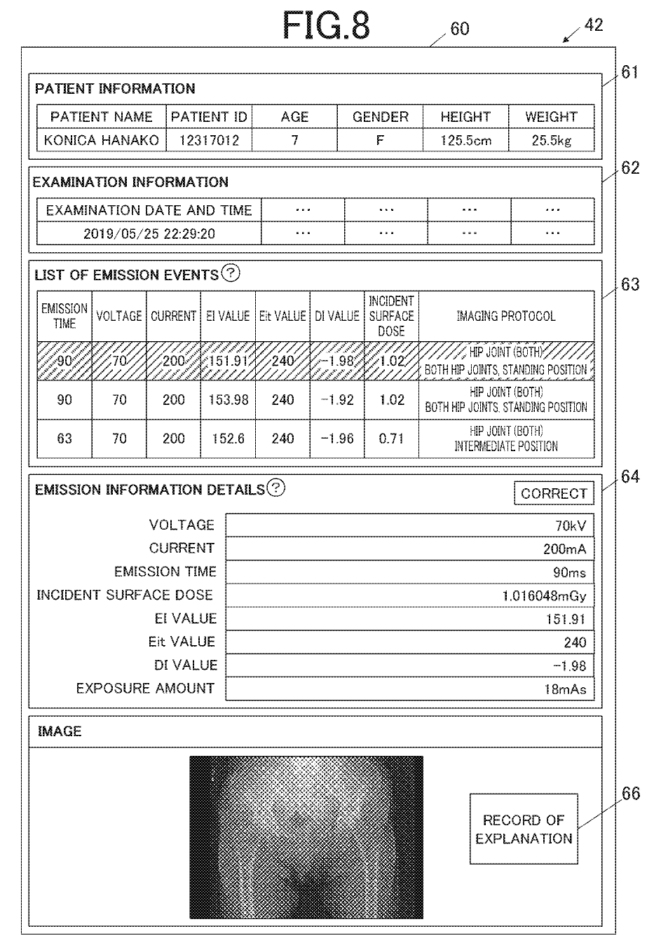

Upon receiving the image data on the dose information management screen for the subject from the dose management apparatus 30 via the communication section 45, the controller 41 of the client terminal 40 displays the dose information management screen based on the received image data on the display part 42 (Step S33). FIG. 8 is a view illustrating an example of a dose information management screen 60 displayed on the display part 42 of the client terminal 40 according to the present embodiment. The dose information management screen 60 is provided with, for example, a patient information region 61, an examination information region 62, an emission event list region 63, an emission information detail region 64, and a radiographic image region 65. Items related to the dose information displayed on the dose information management screen 60 is an example, and items other than those displayed on the dose information management screen 60 illustrated in FIG. 8 may be displayed.

In the patient information region 61, information on the examination target in the selected emission event is displayed. In the examination information region 62, information on the examination to which the selected emission event belongs is displayed. In the emission event list region 63, a list of emission events associated with the examination is displayed. In the emission information details region 64, dose information of the emission event highlighted in the emission event list region 63 is displayed. In the radiographic image region 65, a radiographic image generated in the emission event is displayed.

In the present embodiment, a display button 66 for displaying gonadal protection explanation information is provided in a region adjacent to the radiographic image region 65. When the display button 66 is selected by the user, a gonadal protection explanation information screen 70 is displayed on the dose information management screen 60. The gonadal protection explanation information screen 70 may be superposed and displayed on the dose information management screen 60 as a separate screen, or may be displayed in the dose information management screen 60 together with other dose information at the same time. Further, the above-described display button 66 may not be provided, and the gonadal protection explanation information screen 70 may be displayed at the same time as the dose information management screen 60 is displayed.

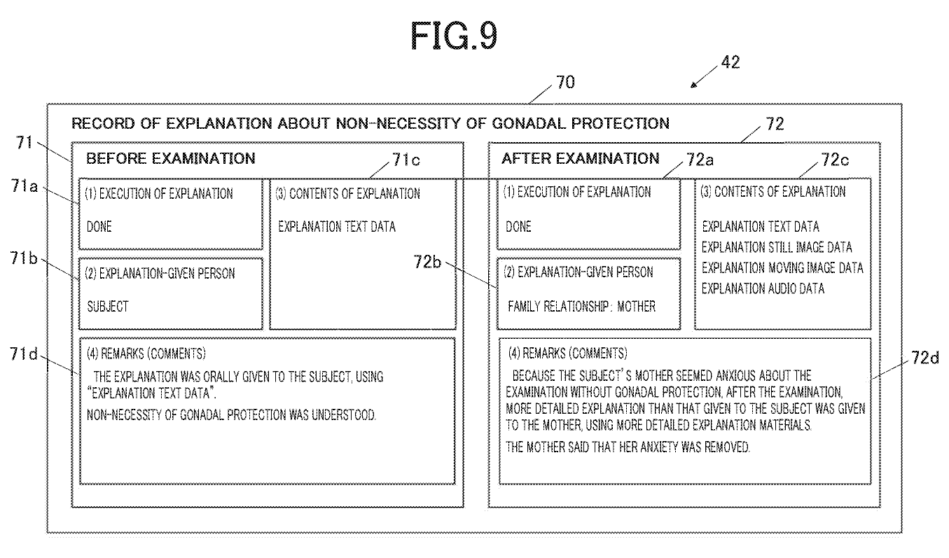

FIG. 9 is a view illustrating an example of the gonadal protection explanation information screen 70 displayed on the display part 42 of the client terminal 40 according to the present embodiment. The gonadal protection explanation information screen 70 is provided with a pre-examination region 71 for displaying pre-examination gonadal protection explanation information and a post-examination region 72 for displaying post-examination gonadal protection explanation information. The pre-examination region 71 is provided with, for example, an explanation execution display section 71a for displaying whether the explanation about gonadal protection has been made. The pre-examination region 71 is provided with a target display section 71b for displaying whether or not the explanation about gonadal protection has been given to a person other than the subject and a relationship between the person who has received the explanation and the subject. The pre-examination region 71 is provided with a content display section 71c for displaying the content used for the explanation about the gonadal protection. The pre-examination region 71 is provided with a comment display section 71d for displaying comments such as a situation at the time of giving the explanation about gonadal protection to the subject or the like. Similarly to the pre-examination region 71, the post-examination region 72 is provided with an explanation execution display section 72a, a target display section 72b, a content display section 72c, and a comment display section 72d.

In the example illustrated in FIG. 9, the explanation about gonadal protection has been given to the subject and his/her guardian before and after the examination. Therefore, on the gonadal protection explanation information screen 70, the gonadal protection explanation information is recorded in both the pre-examination region 71 and the post-examination region 72. In a case where the explanation about gonadal protection has been given to the subject and his/her guardian at before or after the examination, the gonadal protection explanation information is recorded in one of the pre-examination region 71 and the post-examination region 72.

According to the present embodiment, the gonadal protection explanation information input in each input section of the explanation information input screen 50 can be recorded in the storage section 32 of the dose management apparatus 30 in association with the radiographic image, the RDSR, and the like of the subject. Furthermore, regarding the gonadal protection explanation information recorded in the dose management apparatus 30, the gonadal protection explanation information screen 70 can be displayed on the display part 42 of the client terminal 40 or the like in response to a display request to the dose management apparatus 30. Thus, the gonadal protection explanation information on each patient can be left as history information, and whether or not the explanation about gonadal protection has been made can be viewed and checked on the gonadal protection explanation information screen 70. As a result, according to the dose management apparatus 30, it is possible to accurately manage various kinds of information such as whether or not the explanation about the gonadal protection has been given to the patient. Further, in a case where the user such as the radiological technologist looks at the contents of the gonadal protection explanation information screen 70 and finds that the user forgets to give the explanation about gonadal protection to the patient or the like, the user can give the explanation about gonadal protection to the patient or the like. Thus, the patient or the like can be relieved of anxiety about exposure to radiation. Further, when addition by the explanation about gonadal protection is enacted with respect to children’s exposure in the future, insurance points can be calculated by using the gonadal protection explanation information such as “done” displayed on the gonadal protection explanation information screen 70. That is, the gonadal protection explanation information displayed on the gonadal protection explanation information screen 70 can be used as evidence that the explanation about gonadal protection has been given to the patient or the like.

Although some preferred embodiments of the present disclosure have been described in detail with reference to the accompanying drawings, the technical scope of the present disclosure is not limited to these examples. Furthermore, those to which various changes/modifications and improvements have been applied naturally belong to the technical scope of the present disclosure within the range of the technical idea described in claims. For example, in the above-described embodiments, the examination apparatus 10 is an X-ray imaging apparatus, but not limited thereto. The examination apparatus 10 may be, for example, a CT apparatus or the like that uses radiation.

In the above-described embodiments, the gonadal protection explanation information includes information on whether or not the explanation about gonadal protection has been given to the patient, and comments such as the situation when the explanation about gonadal protection has been given to the subject or the like, and the like. As a modification example of the above-described embodiment, the gonadal protection explanation information may include consent information indicating that the patient has consented to the examination without using a gonadal protector. The consent information preferably includes an identifier such as a name for identifying a person who has given consent, a relationship between the person who has given consent and the subject, and a signature of the person who has given consent.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A dose management apparatus that manages radiation dose information on an examination of a subject, the dose management apparatus comprising a hardware processor that records whether an explanation about gonadal protection has been made for execution of an examination.

2. The dose management apparatus according to claim 1, wherein the hardware processor records whether the explanation about the gonadal protection has been made before the execution of the examination.

3. The dose management apparatus according to claim 1, wherein the hardware processor records whether the explanation about the gonadal protection has been made after the execution of the examination.

4. The dose management apparatus according to claim 1, wherein the hardware processor records whether the explanation about the gonadal protection has been given to a person other than the subject and a relationship between the person who has received the explanation and the subject.

5. The dose management apparatus according to claim 1, wherein the hardware processor records a content of the explanation about the gonadal protection.

6. The dose management apparatus according to claim 1, further comprising an inputter with which a comment on the making of the explanation about the gonadal protection is input,
wherein the hardware processor records the comment input with the inputter.

7. The dose management apparatus according to claim 1, wherein the hardware processor outputs recorded information on the explanation about the gonadal protection.

8. The dose management apparatus according to claim 7, wherein the hardware processor outputs the information on the explanation about the gonadal protection and exposure amount information in the examination.

9. The dose management apparatus according to claim 8, wherein the exposure amount information includes a reference value of an exposure dose for an age and a weight of the subject.

10. The dose management apparatus according to claim 8, wherein the reference value of the exposure dose is determined based on an incident surface dose or an area dose.

11. A dose management method of a dose management apparatus that manages radiation dose information on an examination of a subject, the dose management method comprising recording whether an explanation about gonadal protection has been made for execution of an examination.

12. A non-transitory computer-readable storage medium storing a program causing, of a dose management apparatus that manages radiation dose information on an examination of a subject, a computer to record whether an explanation about gonadal protection has been made for execution of an examination.
